(19) United States
(12) Patent Application Publication
(10) Pub. No.: US 2007/0134648 A1
(43) Pub. Date: Jun. 14, 2007

(54) Affinity-based enrichment of phosphorylated peptides and/or proteins

(75) Inventor: Vukic Soskic, London (GB)

Correspondence Address: SEED INTELLECTUAL PROPERTY LAW GROUP PLLC, 701 FIFTH AVE, SUITE 5400, SEATTLE, WA, 98104, US

(73) Assignee: Vukic SoSKIC and Andre Schrattenholz

(21) Appl. No.: 10/555,703

(22) PCT Filed: Apr. 27, 2004
(86) PCT No.: PCT/EP04/04405

§ 371 Date: Dec. 18, 2006

(30) Foreign Application Priority Data
May 6, 2003 (DE) 103 21 901.3

(51) Int. Cl. C12Q 1/00; C07K 14/47; C07F 15/00

(52) U.S. Cl. 435/4; 530/412; 530/352; 548/101; 546/2

(57) ABSTRACT

The invention relates to a substance comprising a solid carrier that is connected to a spacer by means of a linker, said spacer comprising at least two defined groups. Said substance is suitable for using as an affinity material for enriching and/or isolating phosphorylated peptides and/or proteins. The inventive substance especially enables tyrosine-phosphorylated peptides and/or proteins to be enriched and/or isolated.

The invention relates to a novel material which is suitable for the enrichment and/or isolation of phosphorylated peptides and/or proteins. Besides the material or the substance, the invention relates to a method for the enrichment and/or isolation of phosphorylated peptides and/or proteins and to a method for preparing the substance.

The phosphorylation and dephosphorylation of proteins in the cell is crucial for the function of many biological systems. Signals are often transmitted into the organism and, in particular, numerous enzymatic activities are controlled by the phosphorylation and dephosphorylation of proteins. Phosphorylations are therefore a crucial factor for signal transduction chains in living cells.

Research on phosphorylated proteins is thus of particular interest. Within the framework of proteomic research, the term “phosphoproteome” was coined in this connection to describe the investigation of essentially all the phosphorylated proteins of a cell. There has in recent years been further development in phosphoproteomic research in particular, because various enrichment protocols for phosphoproteins or phosphopeptides have been optimized, fractionation methods have been improved and, in particular, multidimensional chromatography has been further developed, in order thus to be able to make the phosphoproteins, which are generally present only in very low concentration, available for analysis for the very first time.

Methods employed to date for the enrichment and identification of phosphorylated proteins usually include a radiolabeling with ³²P-labeled ATP and subsequent SDS polyacrylamide gel electrophoresis or thin-layer chromatography. An Edman sequencing may also be carried out to identify the phosphorylated proteins.

A general problem in the investigation of proteins involved in signal transmission cascades, like in particular the phosphorylated proteins, is that these proteins are generally expressed only in very small quantity and the stoichiometry of the phosphorylation is generally relatively low. Traditional methods for investigating and, in particular, for identifying these proteins are therefore often not suitable, since the quantities of protein necessary therefor can be provided only with very great difficulty.

Because of its particular sensitivity, versatility and speed, mass spectrometry has proved to be a very suitable method for investigating phosphorylations. However, various studies have shown that the ion signal caused by phosphorylated peptides is significantly depressed by the presence of nonphosphorylated peptides. It is therefore necessary for the phosphorylated proteins or peptides to be further enriched in relation to the nonphosphorylated proteins or peptides, in order thus to be able to improve the detectability of the phosphorylated sites.

A conventional method for enriching phosphoptoteins uses phospho-specific antibodies for an affinity purification of the phosphorylated proteins or peptides. The use in particular of antiphosphotyrosine antibodies has proved to be successful in this connection, whereas the use of antibodies against phosphoserine- or phosphothreonine-containing proteins has not as yet been described so often. An alternative to enrichment by antibodies is the use of immobilized metal affinity chromatography (IMAC). This method is based on the affinity of the negatively charged phosphate groups of the proteins to be enriched for positively charged metal ions such as, for example, Fe³⁺or Ga³⁺, which are immobilized on a chromatographic support. IMAC has already been employed in combination with electrospray ionization (ESI) tandem mass spectrometry (Stensballe et al., Proteomics 1 (2001), 207-222) or matrix-assisted laser desorption/ionization (MALDI) mass spectrometry (MS) and alkaline phosphatase treatment for determining the phosphorylation sites (Raska et al., Anal. Chem. 74 (20.02), 3429-3433).

The advantage of these conventional methods is that the entire phosphoproteome of a cell can be isolated thereby. However, particular problems arise especially in the investigation of phosphotyrosine-containing proteins or peptides, because the content of phosphotyrosine in proteins in, for example, vertebrate cells is only about 0.05% compared with the content of phosphoserine (about 90%) and phosphothreonine (about 10%).

Ojida et al. (J. Am. Chem. Soc. 124 (2002), 6256-6258) describe a fluorescent chemosensor which interacts with a phosphorylated peptide surface for the investigation of tyrosine-phosphorylated proteins. The authors were able to show that anthrazine derivatives having two zinc(II) dipicolylamine residues selectively bind phosphorylated peptides and thus bring about a change in the fluorescence spectrum. It is possible with such a fluorescent chemosensor to detect phosphorylated peptides in aqueous solution. The anthrazine derivatives described therein show particular specificity for phosphotyrosine-containing peptides. Mito-oka et al. (Tetrahedron Letters. 42 (2001), 7059-7062) report that similar components are able to bind dihistidine sequence motifs of peptides.

Since tyrosine phosphorylations in the living cell are particularly important especially in signal, transduction and in other regulatory mechanisms, the object of the invention is to provide a method which is an improvement over conventional methods and with which peptides and/or proteins phosphorylated on tyrosine can be enriched and/or isolated.

This object is achieved by a substance as described in claim 1. Claim 8 describes an enrichment or isolation method which employs a corresponding substance. Claims 11, 13 and 15 are concerned with the use of this substance and with a corresponding affinity material. Claim 16 claims a method for preparing the substance of the invention. The dependent claims relate to various preferred embodiments of these aspects of the invention. The wording of all the claims is hereby included in the description by reference.

The invention provides a substance which is suitable as affinity material for the enrichment and/or isolation of phosphorylated peptides and/or proteins. This substance comprises a solid support which is connected via a linker to a spacer. This spacer has at least two groups which are described by the following general formula: 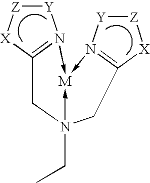
The meanings in this formula are
X, Y: CR′, N, S and/or O;
Z: CR″₂ and/or (CR″)₂;
R′, R″: H, alkyl, halogenyl and/or O-alkyl; and
M: Mn²⁺, Fe³⁺, Co²⁺, Ni²⁺, Cu²⁺, Zn²⁺, Al³⁺ and/or Ga³⁺.

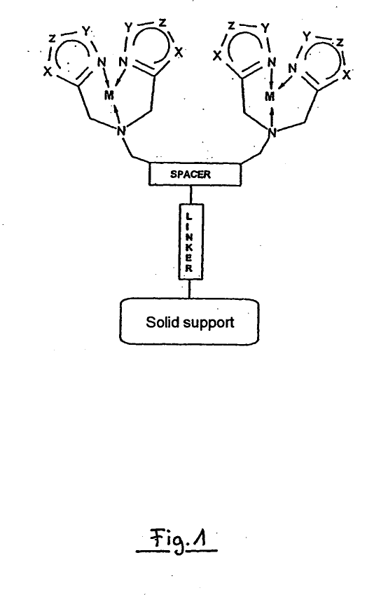

For illustration, the substance of the invention is depicted graphically in FIG. 1. The various letters therein stand for the meanings already described.

This substance and, in this connection, especially the spacer having these at least two groups has a high affinity for phosphorylated peptides and/or proteins. This substance binds corresponding peptides and/or proteins with high affinity and therefore makes it possible to enrich and/or isolate these peptides and/or proteins from a sample. Immobilization of this high-affinity component makes it possible to employ the substance as affinity material which is used for example like a conventional column chromatography material. This substance can therefore be used with protocols like those directly evident to the skilled worker for the enrichment and/or isolation of phosphorylated peptides and/or proteins from a sample. Zinc complexes of the substances of the invention have proved to be particularly advantageous.

The substance of the invention is particularly suitable for the enrichment or isolation of phosphorylated peptides and/or proteins which are phosphorylated on one or more tyrosine residues (pTyr). The substance of the. invention or the substances can therefore also be described as synthetic pTyr receptors. In a preferred embodiment of the substance of the invention, the group is a derivative of 2,2′-dipicolylamine. A corresponding substance of the invention has a particularly high affinity for tyrosine-phosphorylated peptides and/or proteins.

In a preferred embodiment of the substance of the invention, the spacer comprises one or more aromatic rings. These are in particular mono-, bi- and/or tricyclic aromatic rings. The spacer is advantageously a dimethylbenzene acid methyl ester which may also have further radicals. In a particularly preferred embodiment, the starting compound for the spacer is characterized by the following formula: 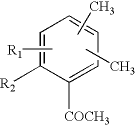
the meanings herein are
R₁, R₂: H, alkyl, halogenyl, O-alkyl and/or mono- or bicyclic aromatic rings.

In very particularly preferred embodiments, the spacer is 2,5-, 3,4- and/or 3,5-dimethylbenzene acid methyl ester or a corresponding derivative.

The linker between the spacer and the solid support is preferably at least one amide, ester, carbamoyl, ether and/or thioether linkage. The specific design of the linker or of the linker compound of course depends on the choice of the support material and the composition of the spacer.

In a very particularly preferred embodiment of the substance of the invention, the spacer including the at least two groups is 2,5-, 3,4- and/or 3,5-bis[(2,2′-dipicolylamino)methyl]benzene acid or the corresponding benzene acid methyl ester.

The solid support is preferably glass, silicate, gold and/or at least one organic polymer. It is generally possible in principle to employ therefor all conventional support materials suitable for chromatographic applications. Advantageous examples are chromatography materials in bead form like those normally employed for column chromatography. Preferred examples thereof are Fraktogel or Sepharose, especially Sepharose 4B. The choice of the support material is, however, not confined to those materials which can be employed for column chromatography. On the contrary, the invention also encompasses materials like those suitable for other affinity enrichment methods. For example, the support may be a material suitable for thin-layer chromatography.

The invention additionally encompasses a method for the enrichment and/or isolation of phosphorylated peptides and/or proteins from a sample. In this connection, sample generally means a sample of biological material. This may comprise for example a cell extract, a tissue extract or the like. It is possible in particular for all proteins of cells to be worked up from a cell culture for this purpose. On the other hand, an appropriate sample may also derive for example from working up biopsy material and/or from particular tissue from organs. On the other hand, it is also possible to employ samples of plant origin, bacterial samples or samples from fungi in the method of the invention. The samples may in this connection be employed either without further purification, so that for example only the cell extract freed of cell detritus is used. On the other hand, the sample may also be further purified. However, it is particularly preferred to use samples without further purification, because it is possible in this way for all phosphorylated peptides and/or proteins of interest in these samples to be enriched and/or investigated in detail, as is particularly of interest in the area of proteomic research.

In the method of the invention, firstly the sample is brought into contact with a substance as has already been described, so that interactions are possible between the substance and the phosphorylated peptides and/or proteins from the sample. In a further step, unbound material, i.e. in particular nonphosphorylated peptides and/or proteins, are removed from the substance. This can take place in particular by washing with suitable buffer solutions. Subsequently, the phosphorylated peptides and/or proteins, i.e. the peptides and/or proteins interacting with the substance, are eluted, i.e. separated from the substance. This again also advantageously takes place through choice of suitable buffer conditions and/or by changing the temperature or the like. This elution step need not necessarily be carried out. It may, especially depending on the chosen analytical method or generally on the objective at which the method of the invention is aimed, be advantageous not to remove the phosphorylated peptides and/or proteins from the affinity material. A suitable protocol for carrying out the method and in particular the choice of suitable buffer conditions will be evident to the skilled worker in this field.

The fraction(s) eluted after carrying out this method now contain the enriched or isolated phosphorylated peptides and/or proteins from the sample. These peptides and/or proteins can subsequently be processed further as required or further purified. This can be achieved for example by carrying out the method of the invention repeatedly or else by other purification methods.

It is particularly preferred for the phosphorylated and preferably eluted peptides and/or proteins subsequently to be analyzed and, where appropriate, identified. This can take place by conventional methods, in particular by one- and/or two-dimensional polyacrylamide gel electrophoresis and/or mass spectrometric methods. It is particularly advantageous to analyze the enriched or isolated phosphorylated peptides and/or proteins by mass spectrometry methods. Particularly preferred in this connection is an electrospray ionization (ESI) tandem. mass spectrometry or the so-called matrix-assisted laser desorption/ionization (MALDI) mass spectrometry. These methods in particular have already proved to be particularly suitable for investigating phosphorylated peptides and/or proteins. The invention also of course encompasses other analytical methods which will be evident to the skilled worker.

The method of the invention is preferably characterized in that the phosphorylated peptides and/or proteins to be enriched are phosphorylated one or more times on one or more tyrosine residues. The specificity of the substance of the invention for tyrosine-phosphorylated peptides and/or proteins and for peptides and/or proteins phosphorylated on another site is governed in particular by the choice of the at least two groups which are located on the spacer shown in FIG. 1. In contrast to threonine- and/or serine-phosphorylated peptides and/or proteins, in particular tyrosine-phosphorylated peptides and/or proteins are involved in signal transduction and regulatory processes in the cell. They are therefore of special biological interest. The problem of conventional methods for investigating tyrosine-phosphorylated peptides and/or proteins is in particular that the tyrosine-phosphorylated peptides and/or proteins are present in the cell only in exceptionally low concentration by comparison with peptides and/or proteins phosphorylated at another site. It is now possible by the method of the invention in particular to enrich or isolate specifically these particularly rare phosphorylated peptides and/or proteins, and thus make them available for investigation. The method of the invention additionally makes it possible, through the specific enrichment or isolation of all tyrosine-phosphorylated peptides and/or proteins of a cell, to provide an overview of these very important control points in the cell, as is the aim in particular of phosphoproteomic research.

The invention additionally encompasses the use of the substance of the invention, as has already been described, as affinity material for the enrichment and/or isolation of phosphorylated pept ides and/or proteins. The peptides and/or proteins to be enriched or isolated are in particular tyrosine-phosphorylated peptides and/or proteins. Reference is also made to the above description concerning further features of this use according to the invention.

The invention additionally encompasses an affinity material for the enrichment and/or isolation of phosphorylated peptides and/or proteins. Reference is made to the above description in this regard too. This affinity material is in particular characterized in that the affinity material is a column chromatography material. This has the advantage that the process for enriching and/or isolating phosphorylated peptides and/or proteins can thereby be carried out with generally customary protocols from protein purification, it being directly within the competence of a skilled worker to adapt conventional protocols to this particular affinity material. Besides column chromatography materials, the affinity material can of course also be designed so that it is suitable for other affinity purifications. Thus, the affinity material can be designed for example such that it is. suitable for a thin-layer chromatography or the like.

The invention moreover encompasses a chromatography column which comprises a corresponding affinity material. The chromatography column in this case may be designed so that it is already loaded with the affinity material of the invention. On the other hand, it may also be advantageous for the chromatography column and the affinity material initially to be separate and for the column to be packed in the appropriate dimension as required. Reference is made to the above description also with regard to this chromatography column.

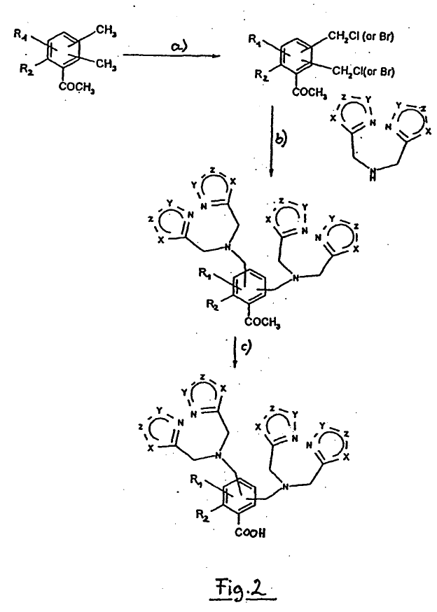

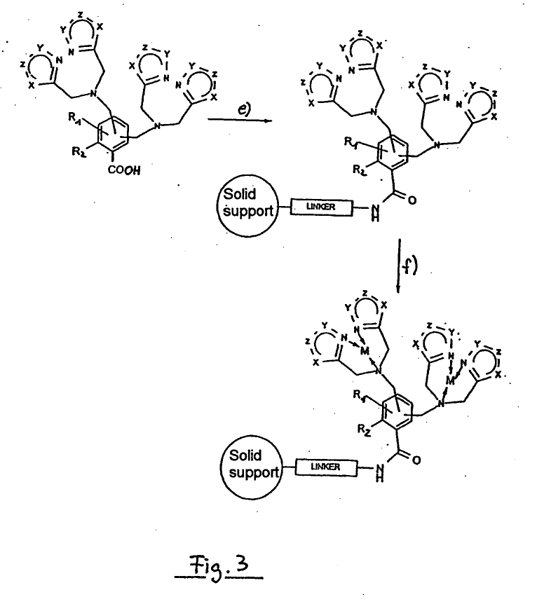

Finally, the invention encompasses a method for preparing a substance which is suitable as affinity material for the enrichment and/or isolation of phosphorylated peptides and/or proteins. This substance has already been described in detail above. The method of the invention for preparing this substance comprises the following method steps, these steps being outlined in FIGS. 2 and 3 for illustration:
a) firstly, at least one compound which comprises at least two methyl radicals and at least one methyl ester residue is reacted with N-bromosuccinimide (NBS), N-bromoacetamide (NBA) and/or SO₂Cl₂ to give the corresponding bromomethyl and/or chloromethyl compound. The compound is preferably the compound V₁ which is characterized by the following formula: 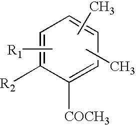
   where the meanings are:
   R₁, R₂: H, alkyl, halogenyl, O-alkyl and/or mono- or bicyclic aromatic rings.
   A particularly preferred representative of this compound V₁ is dimethylbenzene acid methyl ester. The reaction product in this case is bis(bromomethyl)benzene acid methyl ester and/or bis(chloromethyl)benzene acid methyl ester. The spacer of the substance of the invention is formed by this compound or corresponding compounds according to the formula.
b) The reaction product from step a) of the method is reacted with alkali metal carbonates, bicarbonates and/or tertiary organic amines and with at least one compound V₂ of the following formula: 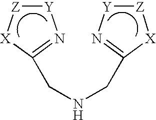
   where the meanings are
   X, Y: CR′, N, S and/or O;
   Z: CR″₂ and/or (CR″)₂;
   R′, R″: H, alkyl, halogenyl and/or O-alkyl.
   If, for example, dimethylbenzene acid methyl ester has been employed in step a) of the method, the reaction product of this method step is bis[(V₂)methyl]benzene acid methyl ester. A particularly preferred representative of V₂ is for example 2,2′-dipicolylamine.
c) The reaction product from step. b) is then reacted with alkali metal hydroxides, carbonates, bicarbonates and/or quaternary ammonium hydroxides to give the corresponding acid. If, for example, dimethylbenzene acid methyl ester was initially employed, the result in this case is bis[(V₂)methyl]benzene acid. A different compound V₁, results, of course, in a different reaction product.
d) As a further step, there is where appropriate activation of a solid support material on which it is intended subsequently to immobilize the reaction product from step c) of the method. Whether an activation of the support is necessary depends on the particular material chosen. The time at which the activation of the support or provision of the support takes place is, of course, not linked to the sequence stated here.
e) The reaction product from step c) of the method is reacted with at least one carbodiimide, uranium and/or phosphonium salt with the support, which is activated where appropriate, so that the reaction product from step c) of the method, i.e. for example bis[(V₂)methyl]benzene acid is immobilized on the support. This combining of support and reaction product takes place via a linker which may be for example an amide, ester, carbomoyl, ether and/or thioether linkage.
f) Finally, the immobilized product from step e) of the method is loaded with metal by treating the mixture with an aqueous solution of Mn²⁺, Fe³⁺, Co²⁺, Ni²⁺, Cu²⁺, Zn²⁺, Al³⁺ and/or Ga³⁺ or the corresponding salts. Particular preference is given in this connection to Zn²⁺ or Co²⁺. The loading with the metal in step f) of the method can also where appropriate be carried out at another time in the reaction sequence.

Further features of the invention are evident from the dependent claims in combination with the figures and examples. The various features can moreover each be implemented on its own or in combination with one another.

The figures show

FIG. 1 diagrammatic representation of the substance of the invention;

FIG. 2 diagrammatic representation of the reaction sequence a) to c);

FIG. 3 diagrammatic reaction sequence of steps e) to f) of the method;.

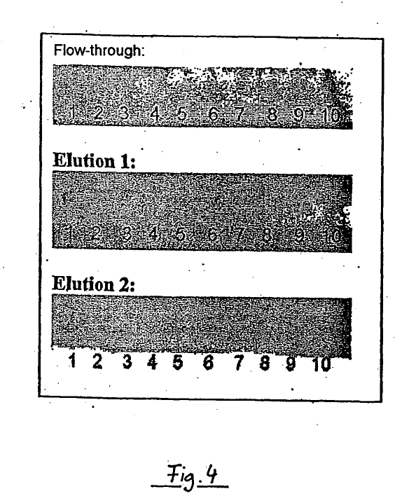

FIG. 4 dot-blot of the proteins eluted from Zn²⁺-BiPy and Co²⁺-BiPy beads. Dot 1: sample 1; dot 2: sample 2; dot 3: sample 3; dot 4: sample 4; dot 5: sample 5; dot 6: sample 6; dot 7: sample 7; dot 8: sample 8; dot 9:sample 9;, dot 10: sample 10;

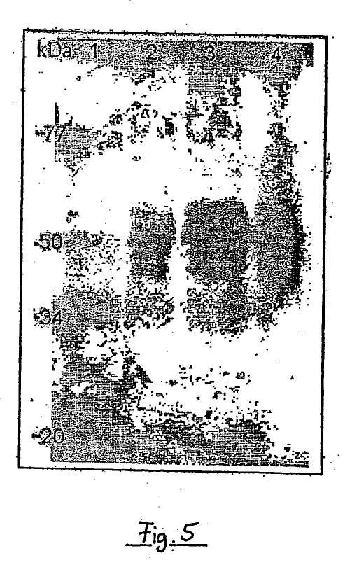

FIG. 5 Western blot of the proteins eluted from Zn²⁺-BiPy and Co²⁺-BiPy beads. Lane 1: total cell extract; lane 2: sample 1 (elution 1); lane 3: sample 3 (elution 1); lane 4: sample 5 (elution 1);

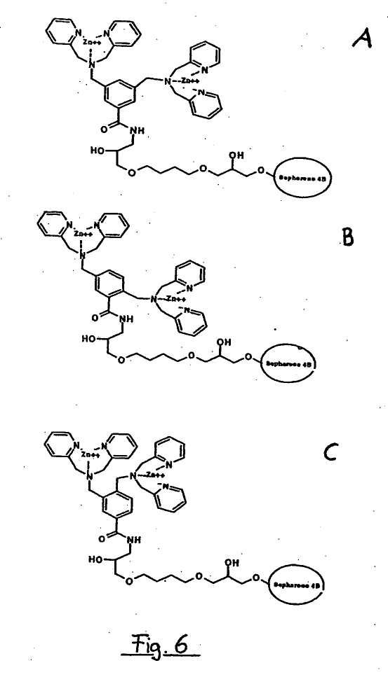

FIG. 6 depiction of preferred embodiments of the substance of the invention. A, B and C show meta, para and ortho variants of the immobilized bis[(2,2′-dipicolylamino)methyl]-benzene acids;

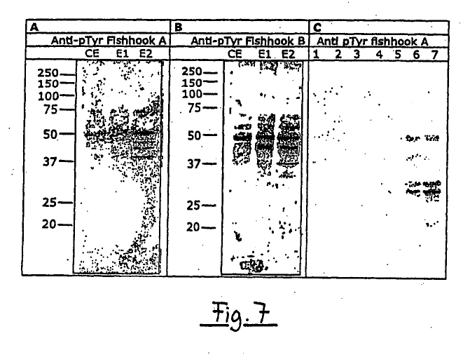

FIG. 7 Western blot analysis of pTyr proteins enriched using the affinity materials A and B. The sample distribution is explained in the examples;

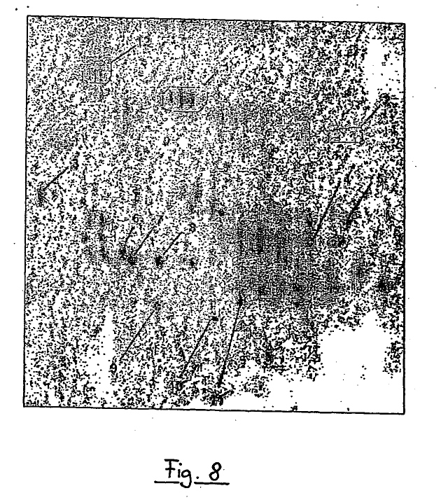

FIG. 8 two-dimensional polyacrylamide gel electrophoresis of the enriched pTyr proteome of endothelin-stimulated fibroblasts of the rat lung.

### EXAMPLE

### A. Preparation of the Affinity Material

### 1. Synthesis of 2,5-, 3,4- and 3,5-di(bromomethyl)benzene acid methyl ester

19.6 g of 2,5-, 3,4- or 3,5-dimethylbenzene acid methyl ester, 47 g of NBS and 0.5 g of benzoyl peroxide were dissolved in 120 ml of carbon tetrachloride, and the reaction was carried out under reflux for 12 h. After cooling, the reaction mixture was filtered, and the solvent was removed by evaporation in vacuo. The resulting dibromo derivatives were purified by recrystallization from n-hexane to result in a yield of 21.2 g of. 2,5-bis(bromomethyl)benzene acid methyl ester with a melting point of 71° C., a yield of 22.2 g of 3,4-bis(bromomethyl)benzene acid methyl ester with a melting point of 74° C., and a yield of 25.3 g of 3,5-bis(bromomethyl)benzene acid methyl ester with a melting point of 78° C.

### 2. Synthesis of 2,5-, 3,4- and 3,5-bis[(2,2.′-dipicolylamino)methyl]benzene acid methyl ester

A solution of KI (1.3 g, 8 mmol) in DMF (8 ml) was added dropwise dropwise over 1 h at 80° C. to a solution of 2,5-, 3,4- or 3,5-bis(bromomethyl)benzene acid methyl ester (2.24 g, 8 mmol), 2,2′-dipicolylamine (3.5 17.2 mmol) and K₂CO₃ (4.42 g, 3.2 mmol) in 20 ml of anhydrous DMF (dimethylfluoride). After stirring for 60 min at 60° C., the reaction mixture was diluted with 1 N HCl and washed twice with ethyl acetate. The aqueous layer was made alkaline with 4 N NaOH and extracted twice with diethyl ether. The combined organic layers were washed with water and alkali, followed by a drying over Na₂SO₄. After removal of the solvent in vacuo, the residue was crystallized from ethanol and resulted in a yield of 3.3 g of 2,5-bis[(2,2′-dipicolylamino)methyl]benzene acid, methyl ester with a melting point of 98° C., a yield of 3.5 g of 3,4-bis[(2,2′-dipicolylamino)methyl]benzene acid methyl ester with a melting point of 102° C. and a yield of 4.2 g of. 3,5-bis[(2,2′-dipicolylamino)methyl]benzene acid methyl ester with a melting point of 111° C.

### 3. Synthesis of 2,5-, 3,4- and 3,5-bis[(2,2′-20 dipicolylamino)methyl]benzene acid

3.0 g of 2,5-, 3,4- or 3,5-bis[(2,2′-dipicolylamino)methyl]benzene acid methyl ester in 50 ml of 80% MeOH were mixed with 1.10 g of Ba(OH)₂ and treated by refluxing under nitrogen for 2 h. A further 0.6 g of Ba(OH)₂ was added, and the refluxing was carried out for a further 2 h. After the reaction mixture had cooled, 50% H₂SO₄ was added in amounts equimolar to Ba(OH)₂. The resulting precipitate was removed by centrifugation, and the remaining solution was evaporated to dryness. Bis[(2,2′-dipicolylamino)methyl]benzene acids were obtained as oily residue which was used further without further purification.

### 4. Activation of the support Material

### 4.1 Activation of Sepharose 4B with 1,4-butanediol diglycidyl ether

50 ml of Sepharose 4B-material which had been sucked dry was transferred into a 250 ml conical bottle which contained 50 ml of 0.6 M NaOH and 50 ml of 1,4-butanediol diglycidyl ether. The suspension was shaken at room temperature for 12 h. The Sepharose 4B material was washed with 2 1 of deionized water and employed immediately for the next reaction step.

### 4.2 Synthesis of amino-1,4-butanediol ether-Sepharose 4B

10 ml of the Sepharose 4B which had been activated with 1,4-butanediol diglycidyl ether and sucked dry was transferred into a 250 ml conical bottle which contained 50 ml of 2.0 M NH₄OH solution. The suspension was shaken at room temperature for 12 h. The Sepharose 4B was washed with 1 l of deioinzed water and used immediately for the next reaction step.

### 4.3 Synthesis of amino-Fraktogel

10 g of epoxy-Fraktogel material were transferred into a 250 ml conical bottle which contained 50 ml of 2.0 M NH₄OH solution. The suspension was shaken at room temperature for 12 h. The Fraktogel material was washed with 1 l of deionized water and used immediately for the next reaction step.

### 5. Immobilization

### 5.1 Synthesis of 2,5-, 3,4- and 3,5-bis[(2,2′-dipicolylamino)methyl]benzene acid-Sepharose 4B

10 ml of amino-1,4-butanediol ether-Sepharose were washed stepwise in a Buchner funnel with 50 ml of 20%, 40%, 60%, 80% and 100% DMF. The amino-1,4-butanediol ether-Sepharose beads were resuspended in 15 ml of DMF, the DMF containing in each case 250 mg of 2,5-, 3,4- or 3,5-bis[(2,2-dipicolylamino)methyl]-benzene acid 20 mg of imidazole, 100 μl of pyridine and 500 μl of diisopropylcarbodiimide were added to this suspension. The suspension was shaken at room temperature for 24 h, and a further 200 μl of diisopropylcarbodiimide were added. After 12 h, the beads were obtained by filtration and washed in the following sequence with 50 ml in each case: 100%, 80%, 60%, 40%, 20% DMF and 500 ml of water. The beads were then resuspended in 20 ml of, 5% NaHCO₃, and 100 μl of acetic anhydride were added. The suspension was shaken at 37° C. for 60 min and then washed with 50 ml of water, 50 ml of 10% acetic acid and 500 ml of water. Finally, the beads were washed in 30% EtOH and stored in a refrigerator.

### 5.2 Synthesis of 2,5-, 3,4- and 3,5-bis[(2,2′-dipicolylamino)methyl]benzene acid-Fraktogel

The synthesis took place in accordance with 5.1, apart from the use of amino-Fraktogel instead of Sepharose 4B.

### 6. Loading of 2,5-, 3,4- and 3,5-bis[(2,2′-dipicolylamino)methyl]benzene acid-Sepharose 4B or Fraktogel with Zn²⁺ or Co²⁺

Columns packed with 2,5-, 3,4- and 3,5-bis[(2,2′-dipicolylamino)methyl]benzene acid-Sepharose 4B or -Fraktogel were washed with 5 volume of water. This was followed by treatment with 3. volume of 0.2 M solution of ZnSO₄ or CoCl₂ in water and then 5 volume of water again. A following equilibration took place with appropriate buffer which was used for the corresponding protein sample preparation.

### B. Enrichment of Tyrosine-Phosphorylated proteins

### Experimental Protocol I

### 1. Isolation of Proteins with Tyrosine Phosphorylations from Rat Liver

The following affinity materials were used in this example:
Material type 1:
2,5-bis[(2,2′-dipicolylamino)methyl]benzene acid-Sepharose 4B (25BiPy-Sepharose 4B)
Material type 2:
3,4-bis[(2,2′-dipicolylamino)methyl]benzene acid-Sepharose 4B (34BiPy-Sepharose 4B)
Material type 3:
3,5-bis[(2,2′-.dipicolylamino)methyl]benzene acid-Sepharose 4B (35BiPy-Sepharose 4B)
Material type 4:

3,5-bis[(2,2′-dipicolylamino)methyl]benzene acid-Fraktogel (35BiPy-Fraktogel)

TABLE 1
Designations of samples in this example

| Sample No. | Material type | Material used |
|---|---|---|
| 1 | 1 | Zn++ |
| 2 | 2 | Zn++ |
| 3 | 3 | Zn++ |
| 4 | 4 | Zn++ |
| 5 | 1 | Co++ |
| 6 | 2 | Co++ |
| 7 | 3 | Co++ |
| 8 | 4 | Co++ |
| 9 | 1 | No metal |
| 10 | Sample without | — |
| (control) | affinity material | |

### 2. Sample Preparation and Isolation of the pTyr protein

### 2.1 Buffers:

1. Lysis buffer: 50 mM NaMOPS, 25 mM NaCl, pH 7.2, 0.5% Zwittergent 3-10, 0.5% CHAPS, 5 mM NaF, 1 mM sodium orthovanadate, 0.2 mM sodium pervanadate, complete mini protease inhibitor cocktail (without EDTA) 1 tabl./10 ml of buffer.
2. Washing buffer: 50 mM NaMOPS, 25 mM NaCl, pH 7.2, 0.1% Zwittergent 3-10, 0.1% CHAPS, 5 mM NaF, 1 mM sodium orthovanadate, 0.2 mM sodium pervanadate.
3. Elution buffer 1: 50 mM NaMOPS, 100 mM NaCl, 50 mM phenyl phosphate (127 mg of phenyl phosphate, disodium salt dihydrate per 10 ml of buffer), pH 7.2, 0.1% Zwittergent 3-10, 0.1% CHAPS, 5 mM NaF, 1 mM sodium orthovanadate, 0.2 mM sodium pervanadate.
4. Elution buffer 2: 50 mM NaMOPS, 100 mM NaCl, pH 7.2, 0.1% Zwittergent 3-10, 0.1% CHAPS, 5 mM NaF, 1 mM sodium orthovanadate, 0.2 mM sodium pervanadate, 50 mM, Na₄EDTA.

### 2.2 Procedure

1. 1 g of rat liver tissue was homogenized with 15 ml of lysis buffer. The suspension was centrifuged in a Sorvall SS34 rotor at 18 000 rpm for 30 min. The supernatant was used and the pellet was discarded. The supernatant was divided into 1.5 ml samples.
2. The samples (1.5.ml) were allowed to run through activated and equilibrated columns which were packed with 0.5 ml of Zn²⁺-BiPy and Co²⁺-BiPy beads.
3. The columns were washed with 3.0 ml of washing buffer, and the first elution was eluted with elution buffer 1 (600 μl) and then with elution buffer 2 (600 μl).

### 3. Dot-Blot Analysis of the Proteins Eluted from the Zn²⁺-BiPy and Co²⁺-BiPy Beads

### 3.1 Buffers:

1. TBS (tris-buffered saline): 10 mM tris-HCl pH 7.4, 170 mM NaCl, 3.4 mM KCl.
2. TBS/Tween: 0.1% Tween 20 in TBS
3. BCIP/NBT (bromochloroindolyl phosphates/-nitroblue tetrazolium): NBT and BCIP stock solutions were stored in a refrigerator for several weeks. The stock solutions were prepared by dissolving 0.5 g of NBT in 10 ml of 70% dimethylformamide. The BCIP stock solution was prepared by dissolving 0.33 g of BCIP disodium salt in 10 ml of DMF in a glass vessel.
4. APB (alkaline phosphatase buffer): 100 mM NaCl, 5 mM MgCl₂, 100 mM Tris-HCl pH 9.5
5. APB/Tween: 0.1% Tween 20 in APB

### 3.2 Procedure

1. The positions of the spots on the nitrocellulose membrane were marked using a soft water-fast pencil. The nitrocellulose membrane was moistened in water and almost completely dried on a clean tissue.
2. The samples were placed on the marked positions, placing 1 μg of protein on each spot.
3. Sufficient quenching buffer (5% BSA (bovine serum albumin) in TBS/Tween) was put in a plastic dish to be covered in such a way that the bottom of the dish was completely covered. The nitrocellulose blot was. cautiously put on the surface of the quenching buffer so that the filter was uniformly moistened. The filter was then submerged in the quenching buffer. Incubation took place with agitation or shaking for at least 2 h. The solution was removed and a solution with the first antibody (1:1000 dilution of pTyr102 antibody, Cell Signaling Technology, # 9416) in 4% BSA in TBS/Tween buffer was added. Incubation took place at 4° C. for 16 h with continuous agitation on a shaker.
4. The antibody solution was poured away. The blot was washed, without allowing the blot to dry, four times with 50 ml of TBS/Tween for 5 min each time. This washing solution was again poured off, and the solution with the second antibody was put on the blot.
5. Incubation with the second antibody (1:5000 dilution of AP-conjugated anti-mouse IgG complete molecule, Sigma 93160 in quenching buffer)-was carried out with agitation at 4° C. for 3 h.
6. This antibody solution was poured off, and the blot was washed four times with 50 ml of TBS/Tween each time, for 5 min each time.
7. The nitrocellulose blot was placed in BCIP/NBT working solution, this solution having been prepared by adding 66 ml of NBT stock solution to 10 ml of APB/Tween with thorough mixing and adding 33 ml of BCIP stock solution.
8. After the color had developed, the reaction was stopped by washing with water and 1% acetic acid.

FIG. 4 shows the results of the dot-blot screening. Therein, dot 1 shows sample 1, dot 2 shows sample 2, dot 3 shows sample 3 etc.

### 4. Western Blot Analysis of the Proteins Eluted from Zn²⁺-BiPy and Co²⁺-BiPy material

4.1 The polyacrylamide gel electrophoresis (PAGE) was carried out by the Laemmli method. In each case 20 μg of protein were loaded in each lane of the 11% PAGE.

### 4.2 Buffers

The same buffers were used as for the dot-blot screening.

### 4.3 Procedure

1. The electroblotting was carried out using a nitrocellulose membrane in accordance with the manufacturer's information using a semidry electroblotting apparatus from BioRad.
2. Sufficient quenching buffer (5% BSA in TBS/Tween) was put in a plastic dish to be covered in such a way that the bottom of the dish was completely covered. The nitrocellulose blot was cautiously put on the surface of the quenching buffer so that the filter was uniformly moistened. The filter was then submerged in the quenching buffer. Incubation took place with agitation or shaking for at least 2 h. The solution was removed and a solution with the first antibody (1:1000 dilution of pTyr102 antibody, Cell Signaling Technology, # 94.16) in 4% BSA in TBS/Tween buffer was added. Incubation took place at 4° C. for 16 h with continuous agitation on a shaker.
3. The antibody solution was poured away. The blot was washed, without allowing the blot to dry, four times with 50 ml of TBS/Tween for 5 min each time. This washing solution was again poured off, and the solution with the second antibody was put on the blot.
4. Incubation with the second antibody (1:5000 dilution of AP-conjugated anti-mouse IgG complete molecule, Sigma 93160 in quenching buffer) was carried out with agitation at 4° C. for 3 h.
5. This antibody solution was poured off, and the blot was washed four times with. 50 ml of TBS/Tween each time, for 5 min each time.
6. The nitrocellulose blot was placed in BCIP/NBT working solution, this solution having been prepared by adding 66 ml of NBT stock solution to 10 ml of APB/Tween with thorough mixing and adding 33 ml of BCIP stock solution.

The results of the Western blot analysis are depicted in FIG. 5. Therein, lane 1 shows the complete cell extract, lane 2 shows sample 1 (elution 1), lane 3 shows sample 3 (elution 1) and lane 4 shows sample 5 (elution 1).

### Experimental Protocol II

### 5. Enrichment of the pTyr Proteins from Rat Brain and Lung Fibroblasts

### 5.1 Buffers:

1. “Zn” lysis buffer: 10 mM imidazole HCl, 40 mM Na MES, 2 μM Zn(SO₄)₂, 0.5% Zwittergent 3-10, 0.5% CHAPS, 5 mM NaF, 1 mM Na₃VO₄, 0.2 mM Na pervanadate, 1×PIC, pH 5.5
2. Washing buffer: 10 mM imidazole HCl, 40 mM Na MES, 100 mM NaCl, 2 μM Zn(SO₄)₂, 0.1% Zwittergent 3-10, 0.1% CHAPS, 5 mM NaF, 1 mM Na₃VO₄, 0.2 mM Na pervanadate, pH 5.5
3. Elution buffer 1: 50 mM Na MES, 50 mM Na phenyl phosphate, 100 mM NaCl, 0.1% Zwittergent 3-10, 0.1% CHAPS, 5 mM NaF, 1 mM Na₃VO₄, 0.2 mM Na pervanadate, pH 5.5
4. Elution buffer 2: 50 mM Na MES, 50 mM Na₂EDTA, 100 mM NaCl, 0.1% Zwittergent 3-10, 0.1% CHAPS, 5 mM NaF, 1 mM Na₃VO₄, 0.2 mM Na pervanadate

### 5.2 Procedure

Minicentrifugation columns from BioRad (# 732-6008) are packed with 600 μl of a 50% suspension of the affinity materials (beads) of the invention. This corresponds in each case to 300 μl of settled beads. On the one hand, immobilized bis[(2,2′-dipicolylamino)methyl]benzene acid was employed as meta variant (beads A) and immobilized bis[(2,2′-dipicolylamino)methyl]benzene acid as para variant (beads B) (see also FIG. 6). The columns were washed in each case 4× with 1 ml of water. The beads were activated with in each case 4×1 ml of 200 mM Zn(SO₄)₂. This was followed by washing 4× with 1 ml of washing buffer. 5-6 mg of protein extract from rat brain or from rat lung fibroblasts were loaded as protein sample on the column. The flow-through was collected and again loaded on. The flow-through was again collected. The columns were washed with 4×1 ml of washing buffer. A first elution was initially carried out with a phenyl phosphate-containing buffer as competition step. A second elution took place with high stringency using EDTA. For the first elution of the bound proteins, the column was eluted with 3×400 μl of elution buffer 1. For the second elution, the column was eluted with 3×400 μl of elution buffer 2. All the eluates were collected. The eluates were concentrated using Biomax K5 (Millipore) to a final volume of about 40-50 μl.

### 6. Analysis of the Fractions

A BCA assay (Pierce) was carried out to determine the proteins in all the fractions.

### 6.1 SDS-PAGE

A BioRad Protean II minigel system (BioRad, Munich) was used to carry out standard SDS-PAGE discontinuous. Laemmli gels. 12% resolving gels with 4% stacking gels were used for all the experiments.

### 6.2 Western blots

Samples (15 μg per sample) of the protein extract, of eluate 1 and of eluate 2 were fractionated on a 12% polyacrylamide gel (BioRad Mini) and blotted onto a PVDF membrane (BioRad).

The membrane was blocked with TBS, 0.1% Tween, 5% BSA for two hours, followed by an incubation with the first antibody (anti-pTyr monoclonal antibody.4G10, Upstate) in a dilution of 1:1000 in blocking buffer overnight. The blot was then washed 3× in TBS/Tween. and subsequently incubated with the secondary antibody (anti-mouse, conjugate with alkaline phosphatase, Sigma) in a dilution of 1:1000 for one hour. The blot was washed 3× with TNS/Tween and developed using the NBT/BCIP substrate (Roche).

### 7. Results

The results prove the enrichment of pTyr proteins by corresponding affinity materials. The immobilized zinc complexes of bis[(2,2′-dipicolylamino)methyl]benzene acid (FIG. 6) were used for this.

FIG. 7 shows the Western blot analysis of pTyr proteins which were enriched using affinity, materials A and B (beads A and B) as shown in FIG. 6. Blots A and B: Western blot analysis of protein extract (CE) from rat brain, eluate 1 (E1) and eluate2 (E2) with in each case the anti-pTyr affinity material A and B. The staining took place with anti-pTyr antibody 4G10 (Upstate). Each lane was loaded with 15 μg of protein.

Blot C from FIG. 7 shows the Western blot analysis of stimulated (EGF, ET1) fibroblasts from the rat lung which were stained with the antibody P-Tyr102. Affinity material A was employed for this. Lane 1: complete proteome of EGF-stimulated cells, lane 2 and 3:. enriched pTyr proteome of unstimulated cells; lane 4: enriched pTyr proteome of ET1 (endothelin)-stimulated cells, eluate .1; lane 5: enriched pTyr proteome of EGF-stimulated cells, eluate 1; lane 5: enriched pTyr proteome of EGF-stimulated cells, eluate 1; lane 6: enriched pTyr proteome of ET1-stimulated cells, eluate 2; lane 7: enriched pTyr proteome of EGF-stimulated cells, eluate. 2. These results show that the currently most widely used antibody for pTyr affinity purifications and detections (4G10) does not recognize some pTyr proteins by comparison with the materials of the invention.

FIG. 8 shows the picture of a two-dimensional polyacrylamide gel electrophoresis of the pTyr proteome of ET1-stimulated fibroblasts from the rat lung which was enriched using affinity material A of the invention. The pattern of the proteins is evident on the silver-stained two-dimensional gel, and many of the tyrosine-phosphorylated proteins were identified by subsequent mass spectrometry. These mass spectrometric analyses prove the enrichment of various tyrosine-phosphorylated proteins such as, for example, a valosine-containing protein with similarities to cdc48 (gi|6678559) from yeast, ATPases, especially the H⁺-transporting two-sector ATPase (gi|92350), the beta-chain of ATPase synthase (gi|114562), actinin alpha 4 (gi|11230802) and others. In addition, previously unknown tyrosine-phosphorylated proteins were also identified.

The Western blot analysis of the pTyr protein enrichment clearly shows that the affinity materials of the invention enrich tyrosine-phosphorylated proteins to a considerable extent from various materials. It was possible to show such an enrichment particularly clearly after stimulation of lung fibroblasts with, for example, endothelin or EGF, both these substances being known to induce tyrosine phosphorylation. In addition, the shown experiments make it clear that the currently used pTyr-specific antibodies cover only a certain fraction of the pTyr proteome. The advantages of the affinity materials of the invention compared with conventional immunological methods therefore derive in particular from the sequence-independent affinity for phosphotyrosine residues, making it possible to cover the complete pTyr proteome better. Moreover, the affinity materials of the invention ensure a greater reproducibility and better flexibility, for example in relation to buffer conditions. Moreover, an analysis can be carried out substantially more quickly, and the costs of such analyses are lower than with conventional methods. Finally, the affinity materials of the invention are particularly advantageously suitable for adaptation to high-throughput processes.

1. A substance comprising a solid support which is connected via a linker to a spacer which has at least two groups of the following formula 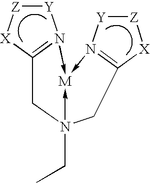
where the meanings are
X, Y: CR′, N, S and/or O;
Z: CR″₂ and/or (CR″)₂;
R′, R″: H, alkyl, halogenyl and/or O-alkyl;
M: Mn²⁺, Fe³⁺, Co²⁺, Ni²⁺, Cu²⁺, Zn²⁺, Al³⁺ and/or Ga³⁺.

2. The substance as claimed in claim 1, wherein the group is formed on the basis of 2,2′-dipicolylamino.

3. The substance as claimed in claim 1, wherein the spacer comprises one or more aromatic rings, in particular mono-, bi- and/or tricyclic aromatic rings.

4. The substance as claimed in claim 1, wherein the spacer is formed on the basis of 2,5-, 3,4- or 3,5-dimethylbenzene acid methyl ester.

5. The substance as claimed in claim 1 wherein the linker comprises at least one amide, ester, carbamoyl, ether or thioether linkage.

6. The substance as claimed in claim 1 wherein the spacer with the at least two groups is formed on the basis of 2,5-, 3,4- or 3,5-bis[(2,2′-dipicolylamino)methyl]benzene acid.

7. The substance as claimed in claim 1 wherein the solid support is glass, silicate, gold or at least one organic polymer, in particular Sepharose or Fraktogel.

8. A method for the enrichment or isolation of phosphorylated peptides or proteins from a sample, comprising the method steps:
contacting the sample with a substance as claimed in claim 1 to form interactions between the substance and phosphorylated peptides/or proteins in the sample,
removing material which does not interact,
where appropriate eluting the phosphorylated peptides or proteins,
where appropriate analyzing the phosphorylated peptides or proteins.

9. The method as claimed in claim 8, wherein the analysis takes place with mass spectrometric methods.

10. The method as claimed in claim 8, wherein the phosphorylated peptides or proteins are tyrosine-phosphorylated peptides or proteins.

11-12. (canceled)

13. An affinity material for the enrichment or isolation of phosphorylated peptides or proteins, in particular of tyrosine-phosphorylated peptides and proteins, comprising at least one substance as claimed in claim 1.

14. The affinity material as claimed in claim 13, wherein the affinity material is a column chromatography material.

15. A chromatography column comprising an affinity material as claimed in claim 13.

16. A method for preparing a substance as claimed in claim 1, comprising the method steps:
a) reacting at least one compound V, of the following formula 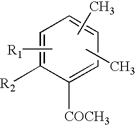
where the meanings are
R₁, R₂: H, alkyl, halogenyl, O-alkyl and/or mono- or bicyclic aromatic rings, with N-bromosuccinimide (NBS), N-bromoacetamide (NBA) and/or SO₂Cl₂;
b) reacting the reaction product from step a) with at least one alkali metal carbonate, bicarbonate or tertiary organic amine with at least one compound V₂ of the following formula 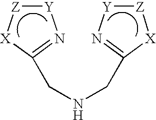
where the meanings are
X, Y: CR′, N, S and/or O;
Z: CR″₂ and/or (CR″)₂;
R′, R″: H, alkyl, halogenyl and/or O-alkyl;
c) reacting the reaction product from step b) with at least one alkali metal hydroxide, carbonate, bicarbonate or quaternary ammonium hydroxide;
d) where appropriate activating a solid support;
e) reacting the reaction product from step c) with at least one carbodiimide, uranium and/or phosphonium salt with the solid support, which is activated where appropriate, to give a reaction product immobilized on the support via amide, ester, carbomoyl, ether or thioether linkages;
f) loading the reaction product from step e) with at least one metal by treatment with an aqueous solution of Mn²⁺, Fe³⁺, Co²⁺, Ni²⁺, Cu²⁺′, Zn²⁺, Al³⁺ or Ga³.

17. The method as claimed in claim 16, wherein the compound V₁ is dimethylbenzene acid methyl ester.

18. The method as claimed in claim 16, wherein the compound V₂ is 2,2′-dipicolylamine.

19. The method as claimed in claim 16, wherein the solid support is glass, silicate, gold or at least one organic polymer, in particular Sepharose or Fraktogel.
